(19) United States
(12) Patent Application Publication
(10) Pub. No.: US 2012/0234333 A1
(43) Pub. Date: Sep. 20, 2012

(54) Medical Fabric With Asymmetrical Layers

(76) Inventors: Shawn E. Jenkins, Duluth, GA (US); Catherine J. Turnbow, Cumming, GA (US)

(21) Appl. No.: 13/049,006

(22) Filed: Mar. 16, 2011

(51) Int. Cl. A61B 19/08; B23B 5/26; D04H 1/70

(52) U.S. Cl. 128/849; 428/212

(57) ABSTRACT

There is provided an SMS laminate for use as a medical fabric. The multilayer non-woven SMS fabric is designed to have the basis weight of the fabric asymmetrically skewed toward one side. In one embodiment the medical fabric is a laminate used as a sterilization wrap having a first spunbond layer, a meltblown layer and a second spunbond layer in which the first spunbond layer is on the side, once an item to be sterilized is wrapped, that is away from the item and that has a greater basis weight than the second spunbond layer that is on the side nearer the item to be sterilized. The medical fabric may also used to produce medical gowns to and drapes.

The present disclosure relates to an improved material and system for fabric that may be used for the sterilization of medical equipment, and to produce medical gowns and drapes.

Medical materials used in surgery are, of course, required to be in a sterile state for use. Many of these items like forceps, scissors, clamps, scalpels, towels, gowns, drapes and the like are reusable and so need to be sterilized prior to reuse. Some of these items are generally disposable or single use items like surgical gowns and drapes and, unless they are pre-packaged in a sterile state, also need to be sterilized by the hospital prior to their single use.

Hospitals have developed protocols for the collection, cleaning and sterilization of materials to be used in surgery. After surgery, the instruments are gathered and sent for cleaning or laundering as necessary, and then sent to a hospital department responsible for sterilization. Sterilization involves placing the items in a stainless steel instrument tray, wrapping the tray with a “sterilization wrap” and sterilizing the package, generally with steam or ethylene oxide, though other methods of sterilization are also sometimes used. After sterilization, the wrapped instrument tray may be taken directly to surgery for use or may be stored for future use. Storage involves the placement of the wrapped tray on a shelf, or on top of another wrapped tray on shelf, in a storage area of the hospital.

Sterilization wrap is most commonly a nonwoven material that is pliable and lightweight, though woven fabrics such as cotton linen are also used. The sterilization wrap functions by allowing the sterilization gas (e.g. steam or ethylene oxide) to pass through the wrap and contact the interior contents of the wrap. It is critical that the sterilization wrap prohibit the passage of microorganisms from the outside of the package to the interior once the wrapped package has been sterilized.

Once the wrapped package has been sterilized it must be transported to surgery or storage, as noted above. It has been found that this transportation and storage result in multiple instances of the sterilized packages being handled and moved. It should be clear that each instance of movement and handling of the sterilized package makes them vulnerable to a breach of the medical fabric and the admission of microorganisms. It is important to keep the handling of sterile packages to a minimum, or baring that, to ensure that the wrap being used is sufficiently durable to resist breach and compromise.

One way to ensure that the package has been wrapped in a sufficiently durable manner is to use a dual layer sterilization wrap. U.S. Pat. No. 5,635,134 to Bourne, et al. discloses a multi-ply sterilization wrap which is formed by joining one or more sheets of sterilization wrap (e.g., two separate sheets or one sheet folded over) together to form two similarly sized, superposed panels that allow convenient dual wrapping of an article. As another example, US patent publication 2001/0036519 by Robert T. Bayer discloses a two ply sterilization wrap that is formed of a single sheet of sterilization wrap material which is folded to form two similarly sized, superposed panels that are bonded to each other. As yet another example, US patent publication 2005/0163654 by Stecklein, et al. discloses a sterilization wrap material that has a first main panel and a second panel that is smaller than the main panel. The second panel is superposed and bonded to the central portion of the main panel such that it is contained entirely within the main panel to reinforce the main panel and/or provide additional absorbency. Still another example is U.S. patent application Ser. No. 12/850,697 that provides a multi-panel sterilization assembly that includes a barrier panel formed of a permeable material, a fold protection panel, and at least one panel attachment means.

Even with the use of multiple ply sterilization wrap fabric, however, breaches in the wrap and contamination of the sterilized items are still possible. The movement of sterilized packages onto shelves and trays has been found to result in abrasion and compression of the wrap. This produces “pressure holes”, very small holes that extend from the outermost layer to the inside of the package. These holes are seen by hospital personnel after the package has been unwrapped, usually by holding the open wrap up to a light. The light can be seen clearly through the holes and indicates a problem in sterility that must be addressed.

In addition to the pressure holes, repeated movement, particularly sliding of the sterilized package, can result in larger holes being formed, especially at the corners of the package. This shear-type of hole is easier to see prior to opening of the package but is nonetheless quite serious and must be rectified and the items re-sterilized prior to use.

In addition to the use of medical fabric as sterilization wrap, surgeons and other healthcare providers often wear an over garment made from medical fabric during operating procedures in order to enhance the sterile condition in the operating room and to protect the wearer. The over garment is typically a gown made from the medical fabric that has a main body portion to which respective sleeves are attached. In order to prevent the spread of infection to and from the patient, the surgical gown prevents bodily fluids and other liquids present during surgical procedures from flowing through the gown. Gowns usually have ties that are used to encircle the wearer and secure the gown around the wearer's body. The attachment point of the tie to the gown has been found to be a weak point, often resulting in the tie being pulled or torn off of the gown as the tie is being used to secure the gown around the body.

Drapes are used to cover a patient during surgery. There are a myriad of different shapes and surgeries that drapes are designed for, but they are generally desired to be impervious to liquid. Drapes are also made from a medical fabric that may be sterilized and that should be free of holes.

One skilled in the art can recognize that despite the improvements in medical fabric that have occurred over the past few decades, the problem of pressure and shear induced holes in medical fabric remains a concern. This results in increased costs for hospitals, patients, insurers and governments because of the required re-sterilization of surgical materials and requires greater time and heightened vigilance from healthcare workers lest unsterile materials be introduced into the sterile surgical setting.

### SUMMARY

The problems discussed above have found a solution to a large degree in the present disclosure, which describes a multilayer non-woven medical fabric wherein the basis weight of particular layers within the fabric is asymmetrically skewed toward one side.

In one embodiment the medical fabric is a laminate having a first (outer) spunbond layer, a meltblown layer and a second spunbond layer (i.e., SMS) in which the first spunbond layer is on the side, once an item to be sterilized is wrapped, that is away from the item and that has a greater basis weight than the second spunbond layer that is on the side nearer the item to be sterilized. In this wrap the laminate desirably has a basis weight of between about 17 and 119 gsm. Other embodiments may have a basis weight between about 34 and 87 gsm and still others may have a basis weight of less than 30 gsm.

The SMS laminate wrap may have its construction skewed so that the first or outer spunbond layer contains between 40 and 80 percent of the basis weight of the laminate. Alternatively, the SMS laminate may have a first spunbond layer that has between 50 and 70 percent of the basis weight of the laminate. The SMS wrap may have a meltblown layer having between 10 and 40 percent of the basis weight of the laminate.

In another embodiment, there is provided a multilayer medical fabric having at least two SMS layers, the first SMS layer on a side away from an item to be sterilized having a basis weight between 17 and 89 gsm and having an asymmetrical construction with an outer spunbond layer having a greater basis weight than an inner spunbond layer. In this construction the term “outer” refers to the spunbond layer away from the item to be sterilized. In this embodiment, the spunbond layer of the second SMS layer having the greater basis weight is on the side away from the item to be sterilized. Alternatively, the spunbond layer of the second SMS layer having the lower basis may be on the side away from the item to be sterilized.

Other objects, advantages and applications of the present disclosure will be made clear by is the following detailed description of a preferred embodiment of the disclosure and the accompanying drawings wherein reference numerals refer to like or equivalent structures.

### BRIEF DESCRIPTION OF THE DRAWINGS

FIG. 1 shows the ternary diagram for a 1.2 osy SMS laminate, overlaid with the least squared fit values of the Simulating Handing test results tested using a 12.5 lb tray.

FIG. 2 shows the ternary diagram for a 1.85 osy SMS laminate, overlaid with the least squared fit values of the Simulated handling test results tested using a 12.5 lb tray.

FIG. 3 shows the ternary diagram for a 1.2 osy SMS laminate, overlaid with the least squared fit values of the Sliding Compression test.

FIG. 4 shows the ternary diagram for a 1.85 osy SMS laminate, overlaid with the least squared fit values of the Sliding Compression test results.

FIG. 5 shows a SMS “construction” ternary diagram. Each point on the diagram represents a particular SMS construction. Note the “symmetric construction line” representing a laminate having spunbond of equal basis weights on either side.

FIG. 6 shows the Sliding Compression results (Y axis) from Table 4, plotted as a function of overall weight percentage of the SMS laminate that is comprised of spunbond oriented toward the probe (X axis). Within this Figure the plus signs represent the data for a 1.85 osy laminate and the circles represent the data for a 1.7 osy laminate. FIG. 6 also shows a linear fit of the data. Parameter estimates of the equation for this line are presented in Table 5.

FIGS. 7A and 7B show cross-sectional views of a co-oriented and counter oriented SMS laminate, respectively.

### DETAILED DESCRIPTION

The typical medical fabric material is a nonwoven fabric or web such as a spunbond, meltblown, spunbond laminate in which the layers are usually produced one onto another, resulting in a sandwich with the meltblown layer in the middle. This is generally referred to as “SMS”.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

The term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. The fibers may also have shapes such as those described in U.S. Pat. No. 5,277,976 to Hogle et al., U.S. Pat. Nos. 5,466,410 to Hills and 5,069,970 and 5,057,368 to Larg man et al., which describe fibers with unconventional shapes.

The term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

A “multilayer nonwoven laminate” means a laminate wherein some of the layers are spunbond and some meltblown such as a spunbond/meltblown/spunbond (SMS) laminate and others as disclosed in U.S. Pat. No. 4,041,203 to Brock et al., U.S. Pat. No. 5,169,706 to Collier, et al, U.S. Pat. No. 5,145,727 to Potts et al., U.S. Pat. No. 5,178,931 to Perkins et al. and U.S. Pat. No. 5,188,885 to Timmons et al. Such a laminate may be made by sequentially depositing onto a moving forming belt first a spunbond fabric layer, then a meltblown fabric layer and last another spunbond layer and then bonding the laminate. Alternatively, the fabric layers may be made individually, collected in rolls, and combined in a separate bonding step. Multilayer laminates generally may also have various numbers of meltblown layers or multiple spunbond layers in many different configurations and may include other materials like films (F) or coform materials, e.g. SMMS, SM, SFS, etc.

SMS laminates used in the production of medical fabrics have been “symmetrical”, meaning that the outer layers of spunbond fabric have had basis weights equivalent to one another. This allows a hospital employee wrapping an item to be sterilized, for example, to use the material without concern about which side is facing downwardly and which fabric is facing the items to be sterilized, except in the case of sterilization wraps that are “sided” for other reasons. While this has simplified the process of wrapping items, this has also resulted in the unanticipated production of shear and pressure holes, as discussed above.

It is desirable that SMS material for use in this disclosure be made in the sequential manner as described above wherein the individual layers are deposited onto a moving forming belt; first a spunbond fabric layer, then a meltblown fabric layer and last another spunbond layer and bonded to form the laminate. As noted above, however, the layers may be made separately, stored for a time in roll form, and unrolled and bonded together to form the laminate in a separate step. In still another alternate method of manufacturing, one or more of the layers (e.g. the spunbond layer) may be made separately and stored as a roll. At a later time the spunbond layer may be unrolled and the other layers (e.g. the meltblown, spunbond) formed and directly deposited onto the spunbond layer.

The disclosures have found that an asymmetrical SMS laminate construction, or one having differing basis weights between the two outer spunbond layers, can reduce the number of pressure and/or shear-induced holes significantly (if oriented appropriately relative to the application of use). In addition, when used as a gown fabric, the asymmetrical SMS can improve the attachment of ties to the gown.

In order to test this proposition, numerous SMS laminates of varying basis weights within the range of those used in sterilization wrap were produced. Symmetrical control fabrics and asymmetrical test fabrics as detailed in Table 1 were tested using two methods; a Simulated Use test and a Sliding Compression test.

The Simulated Use test compares the relative durability of medical fabric materials wherein durability for this test is defined as the resistance to pressure hole formation. Pressure holes can appear as pinholes in wrap fabric which become visible when the wrap is positioned between a strong light and the viewer. It has been found that the wrap exhibiting this mode of failure is characterized as having fused fibers around the edge of the hole. Failures of this type are usually found along the perimeter of the bottom of the sterilization tray or, more particularly, at the corners of the tray. Failure is believed to result when a layer of wrap is sandwiched between two hard surfaces (e.g. the bottom of the tray and a shelf) and when sudden or excessive pressure is applied, pinching the fabric. The Simulated Use test was developed to force failure in medical fabric material via pressure holes. The method does this by simulating aggressive wrap in-use conditions.

The Simulated Use test for wrap is performed, generally, by loading trays with a prescribed amount of weight, ranging from 2.5 lbs to 40 lbs (1.14 to 18.2 kg). The trays are wrapped with the material to be tested and the wrapped trays placed on a sterilization cart having wire mesh shelving. The cart is pushed over a rough surface for a prescribed number of cycles, the trays are unwrapped and the fabrics are inspected under low power magnification for holes at the tray corner regions. A percent failure is calculated utilizing the number of corner regions that failed and then dividing by the total number of corners tested.

More specifically, the Simulated Use test is performed using wire mesh surgical trays and, typically, four sheets of wrap per sample per tray weight are tested (i.e. number of corners per sample per tray weight is sixteen). The preferred tray design is manufactured by Hupfer® Metallwerke GmbH & Co. KG with a 0.5 mm diameter wire base and is approximately 22.5 cm×22.5 cm×5 cm (length×width×height). The cart has a wheel width of 2.54 cm and a wheel diameter of 12.7 cm. The shelves on the cart are comprised of 1/16″ (1.6 mm) diameter wire in the basket weave configuration. The rough floor surface is a No Trax® floor mat from Superior Manufacturing Group, Inc. and is a 3′×6′ (91.4 cm by 182.9 cm) black rubber mat, ⅜″ (0.95 cm) thick. The floor mat has holes with a density of about 20 per square foot (929 square cm) and each hole is rectangular with dimensions of ½″×2″ (1.3 cm by 5.1 cm) with the long dimension of the hole running across the width of the mat.

In order to perform the test, the samples of individual base sheets are cut into rectangles of 18″×22″ (45.7 cm by 55.9 cm), though sheet size can vary if alternate size trays are used. The desired weight is placed in four empty trays and the trays are wrapped making sure that wrap material is snug around the tray base. The corners of the tray should be wrapped using triangular folds. For consistency, if the wrap is excessively baggy around the corners, tape is used to lift and pull the sides of material tight around the base of the tray. The trays are placed on the wire shelf, making sure two corners of the tray, preferably the tray end with folds on the side, are on the wire portion of the shelf. The remaining two corners may hang over the edge of the shelf. Trays are set down on the shelf, not slid onto the shelf, to prevent premature damage. Once the trays are on the shelf, adjustment and sliding of the trays are kept to a minimum.

The cart is rolled at a brisk walking pace over the rough surface. One time over the mat and one time back constitutes one cycle over the rough surface. Rolling of the cart over the rough surface is repeated for five cycles. Upon completing five cycles, trays are rotated 180 degrees such that the tray corners that hung off the shelf now are on the wire mesh shelf and the five cycles repeated. The trays are unwrapped and removed from the cart. The wrap samples are then evaluated for holes using low power optical magnification (i.e. microscope with approximately 3× magnification). To help enhance the visualization of holes/tears, a backlight is used. The number of failures observed is recorded along with the total number of corners tested, generally sixteen. A percentage failure is then calculated for a particular sample at a given tray weight.

In the Sliding Compression test a weighted probe is placed on a sample of medical fabric and the probe with the weight resting on it is slid a specified distance. The tendency of the wrap to develop holes can be determined from a representative sample.

In order to perform the Sliding Compression test, 50.8 by 152.4 mm (2 by 6 inch) specimens are cut from a sample. They should be inspected to ensure they are free of cuts, folds, wrinkles, etc. A weighted probe having a predetermined weight placed on it is placed on the wrap. In the case of asymmetric samples, the probe may be placed on the “light” spunbond side or the “heavy” spunbond side. In the case of symmetric constructions, the probe was placed against either side during testing. However, within a sample set the same side of the symmetric samples was used consistently. The weighted probe is moved forward and backwards for one cycle. If a hole is formed, weight is reduced by a predetermined increment (typically 50 g) and the test repeated. If a hole is not formed, weight is increased by the same predetermined increment and the test repeated. The test is repeated until ten of the last 20 cycles results in a failure. The average weight used over those cycles is then recorded.

TABLE 1
Asymmetric and Symmetric SMS sample
details and test methods performed.

| | | | Basis | Simulated | Sliding |
|---|---|---|---|---|---|
| | | S/M/S | Weight | Use Test | Compression |
| | S/M/S (osy) | (percentage) | (osy) | Performed? | Performed? |
| 1 | 0.76/0.33/0.76 | 41/18/41 | 1.85 | yes | yes |
| 2 | 0.63/0.59/0.63 | 34/32/34 | 1.85 | yes | yes |
| 3 | 0.43/0.33/1.09 | 23/18/59 | 1.85 | yes | yes |
| 4 | 0.38/0.29/1.18 | 20/16/64 | 1.85 | yes | yes |
| 5 | 0.43/0.34/0.43 | 36/28/36 | 1.20 | yes | yes |
| 6 | 0.30/0.34/0.56 | 25/28/47 | 1.20 | yes | yes |
| 7 | 0.30/0.60/0.30 | 25/50/25 | 1.20 | yes | yes |
| 8 | 1.33/0.26/0.26 | 72/14/14 | 1.85 | yes | yes |
| 9 | 2.06/0.26/0.26 | 80/10/10 | 2.58 | yes | yes |
| 10 | 0.26/1.33/0.26 | 14/72/14 | 1.85 | yes | yes |
| 11 | 0.26/2.06/0.26 | 10/80/10 | 2.58 | yes | yes |
| 12 | 0.38/0.29/0.38 | 36/28/36 | 1.05 | yes | yes |
| 13 | 0.48/0.44/0.48 | 34/32/34 | 1.40 | yes | yes |
| 14 | 0.73/0.40/0.73 | 39/22/39 | 1.85 | yes | yes |
| 15 | 0.81/0.43/0.81 | 39/21/39 | 2.05 | yes | yes |
| 16 | 1.04/0.50/1.04 | 40/20/40 | 2.57 | yes | yes |
| 17 | 0.71/0.27/0.36 | 53/20/27 | 1.34 | no | yes |
| 18 | 1.18/0.29/0.59 | 29/14/57 | 2.06 | no | yes |
| 19 | 1.44/0.35/0.72 | 57/14/29 | 2.51 | no | yes |

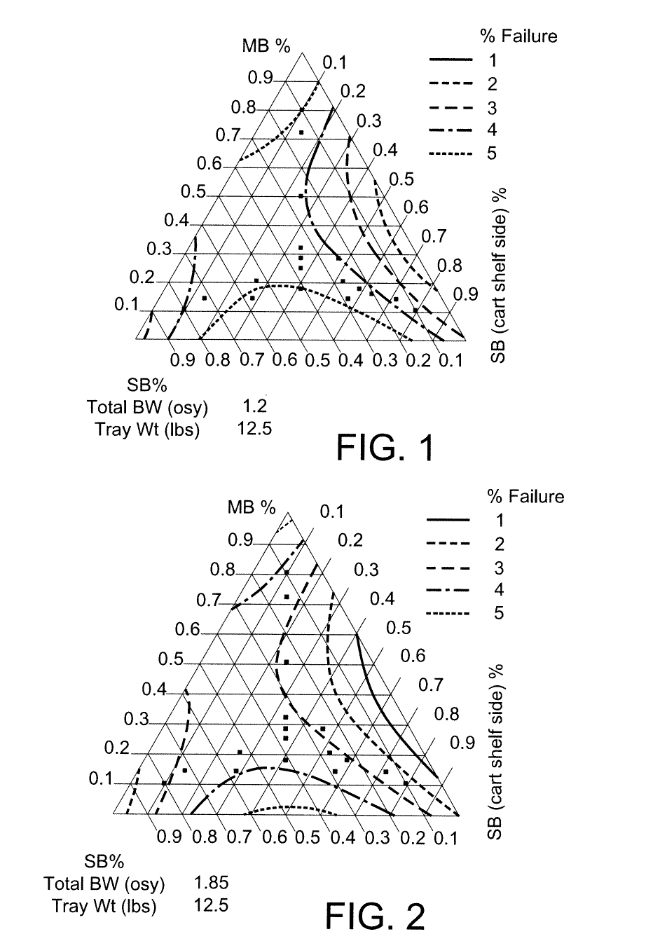

Symmetric and asymmetric SMS samples were obtained and tested as summarized by Table 1. Ternary diagrams were used as a convenient means to illustrate how the results of the Simulated Use and Sliding Compression tests changed as a function of composition as well as how the SMS was oriented. Various ternary diagrams are presented in FIGS. 1 though 6. The effect of sidedness was tested by orienting the heavier spunbond side against the cart shelf or probe as well as orienting the heavier spunbond side away from the cart shelf or probe. Results from these two test methods were collected and input into JMP® 8.0 statistical software. A standard least square fitting method was used to determine statistically significant testing and sample factors, and correlate their effect on Sliding Compression and Simulated Handling test results. A zero-intercept form of the multiple regression model was used since the three factors SB percentage, MB percentage, and SB (cart shelf or probe side) percentage, must sum to 1.

For the Simulated Handling test results, the following factors were used in the standard least square fit of the data;
Total BW (osy)
Tray Wt (lbs)
SB percentage
MB percentage
SB (cart shelf side) percentage
SB percentage×SB (cart shelf side) percentage
MB percentage×SB (cart shelf side) percentage

For the Sliding Compression test results, the following factors were used in the Standard Least Square fit of the data;
Total BW (osy)
SB percentage
MB percentage
SB (probe side) percentage
SB percentage×SB (probe side) percentage
MB percentage×SB (probe side) percentage

Tables 2a and 2b provide summary statistics for the respective fit models. RSquare is a value between 0 and 1 indicating how much of the variation in the response is due to the model and how much is residual error. A value of 1 indicates all variation is accounted for by the model, while 0 indicates no variation is accounted for by the model. The majority of the variation in both data sets is captured by the respective fit models.

TABLE 2a
Summary statistics for fit of Simulated Handling data

| | |
|---|---|
| RSquare | 0.669261 |
| RSquare Adj | 0.64315 |
| Root Mean Square Error | 0.216923 |
| Observations (or Sum Wgts) | 83 |

TABLE 2b
Summary statistics for fit of Sliding Compression data

| | |
|---|---|
| RSquare | 0.869815 |
| RSquare Adj | 0.862333 |
| Root Mean Square Error | 275.0788 |
| Observations (or Sum Wgts) | 93 |

Tables 3a and 3b provide the model parameters used to fit the Simulated Handling and Sliding Compression data, respectively. The prediction formula used to fit the data is the linear combination of the values in the Estimate Column, multiplied by their corresponding terms. The tables also provide a Prob>|t| measure. Prob>|t| is the probability of getting, by chance alone, a t-ratio greater (in absolute value) than the computed value, given the null hypothesis. A value below 0.05 is interpreted as evidence that the parameter is significantly different from zero.

TABLE 3a
parameter estimates for Simulated Handing data

| Term | Estimate | Std Error | t Ratio | Prob > \|t\| |
|---|---|---|---|---|
| Total BW (osy) | −0.466598 | 0.065017 | −7.18 | <.0001* |
| Tray Wt (lbs) | 0.0494607 | 0.005062 | 9.77 | <.0001* |
| SB percentage | 0.3411615 | 0.238441 | 1.43 | 0.1566 |
| MB percentage | 1.2999507 | 0.393658 | 3.30 | 0.0015* |
| SB (cart shelf side) | 0.476491 | 0.345021 | 1.38 | 0.1713 |
| percentage | | | | |
| SB percentage*SB (cart | 3.6083094 | 1.200156 | 3.01 | 0.0036* |
| shelf side) percentage | | | | |
| MB percentage*SB (cart | −2.995546 | 2.250682 | −1.33 | 0.1872 |
| shelf side) percentage | | | | |

TABLE 3b
parameter estimates for Sliding Compression data

| Term | Estimate | Std Error | t Ratio | Prob > \|t\| |
|---|---|---|---|---|
| Total BW (osy) | 1335.0801 | 86.46295 | 15.44 | <.0001* |
| SB percentage | 549.24112 | 254.1128 | 2.16 | 0.0334* |
| MB percentage | −3677.834 | 439.3463 | −8.37 | <.0001* |
| SB (probe side) | −695.5863 | 428.3525 | −1.62 | 0.1080 |
| percentage | | | | |
| SB percentage*SB | −10403.16 | 1050.37 | −9.90 | <.0001* |
| (probe side) | | | | |
| percentage | | | | |
| MB percentage*SB | 10018.813 | 2365.352 | 4.24 | <.0001* |
| (probe side) | | | | |
| percentage | | | | |

From these Tables several variables related to the composition of SMS were found to be statistically correlated to the response variables. In particular, in the case of the Simulated Handling data, MB percentage and the SB percentage×SB (cart shelf side) percentage cross term were determined to be significant, while in the case of the Sliding Compression data, SB percentage, MB percentage, and the two cross terms SB percentage×SB (probe side) percentage and MB percentage×SB (probe side) percentage, were found to be significant.

The Standard Least Squares fit of each data set was plotted as a function of the three compositional components, SB percentage, MB percentage and SB (cart shelf or probe side) percentage using a ternary diagram. Within a ternary diagram, each point represents a unique combination of the three compositional components, in this case MB percentage, SB percentage, and SB (cart shelf side or probe) percentage. The three of these compositional components must sum to 1 (or 100 percent). Overlaid on the ternary diagrams given in FIGS. 1, 2 and 3, 4 are the least squared fit values of the Simulating Handing and Sliding Compression test results, respectively.

FIG. 1 shows the ternary diagram for a 1.2 osy laminate and FIG. 2 shows the ternary diagram for a 1.85 osy laminate, both tested using a 12.5 lb tray. In FIGS. 1 and 2, the percentage of spunbond on the outermost side (cart shelf side) is on the right hand side of the pyramid, the meltblown (center layer) percentage is on the left hand side of the pyramid and the spunbond percentage on the side facing inwardly (away from the cart shelf) is on the bottom of the pyramid. The three percentages add up to 1.0. As an example, point “A” in FIG. 1 represents an SMS laminate having 0.5 percent of the laminate weight on the side away from the cart, 0.4 percent of the weight as meltblown and 0.1 percent as spunbond on the cart facing side.

The lines that are labeled in FIGS. 1 and 2 represent the relative amount of samples that failed the Simulated Handling test, with durability increasing as indicated by the line and chart legend. The increasing failure rate is shown with line 1 representing the lowest failure rate and line 5 representing the highest failure rate. As can be deduced from the diagrams (FIGS. 1 and 2) the most durable laminate in the Simulated Use test is one that has approximately 0.5 to 0.8 spunbond facing the cart, approximately 0.5 to 0.2 meltblown and virtually no spunbond on the side away from the cart. It should be noted that although such a laminate would be very durable, this test does not take into account other desirable features of the laminate and so would probably not actually be used. It is here for illustrative purposes only.

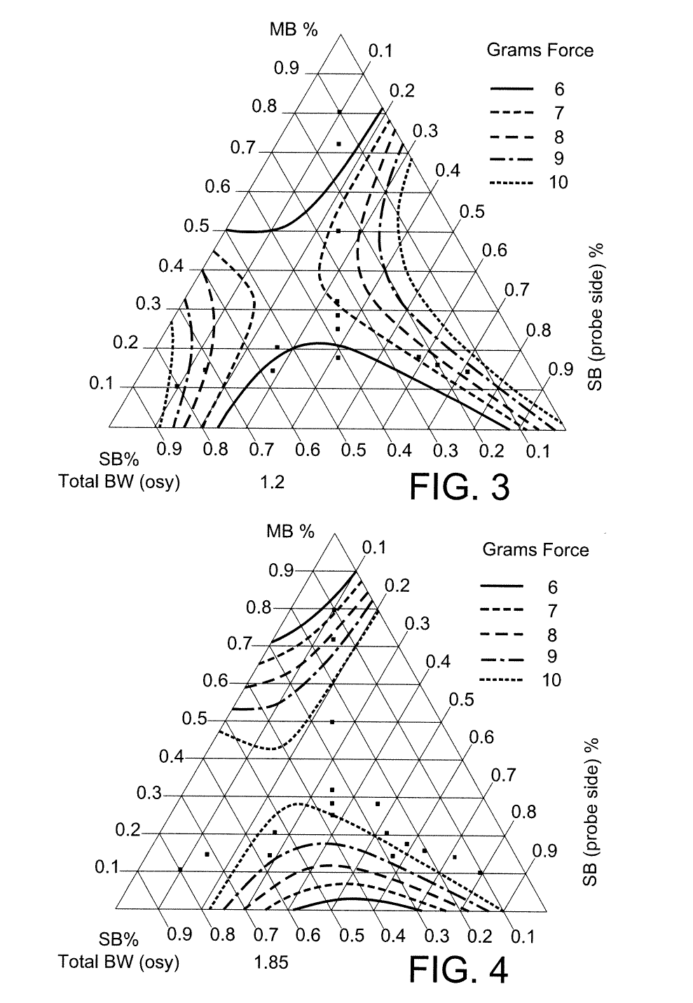

The ternary diagrams shown as FIGS. 3 and 4 represent the predicted grams of force needed to cause a hole via the Sliding Compression test at 1.2 osy and 1.85 osy total basis weight, respectively. In FIGS. 3 and 4, the percentage of spunbond on the outermost side (probe side) is on the right hand side of the pyramid, the meltblown (center layer) percentage is on the left hand side of the pyramid and the spunbond percentage on the side facing inwardly (away from the probe) is on the bottom of the pyramid. The lines represent the relative average amount of weight (grams force) needed to result in a hole in the sample. Grams force was found the change as indicated by line and chart legend. The increasing amount of force required to create a hole is shown with line 6 representing the least amount of force and line 10 representing the greatest amount of force. The three percentages add up to 1.0 in the same manner as in FIGS. 1 and 2.

In the Sliding Compressing test, the greater the grams force needed to produce a hole, the greater the durability. In both data sets, durability was found to be strongly correlated to overall basis weight of the SMS. However, for a given basis weight, several compositional trends can be observed. In general, durability is observed to increase at greater overall SB percentages (lower MB percentage). There is, however, observed to be a lower limit in MB percentage, below which durability is adversely affected. This is believed to be due to the greater coverage efficiency of the MB and because below a critical amount of MB the test methods can no longer differentiate between a hole and interstitial fiber spacing. Furthermore, in practicality MB can only be reduced so far before bacterial barrier is compromised.

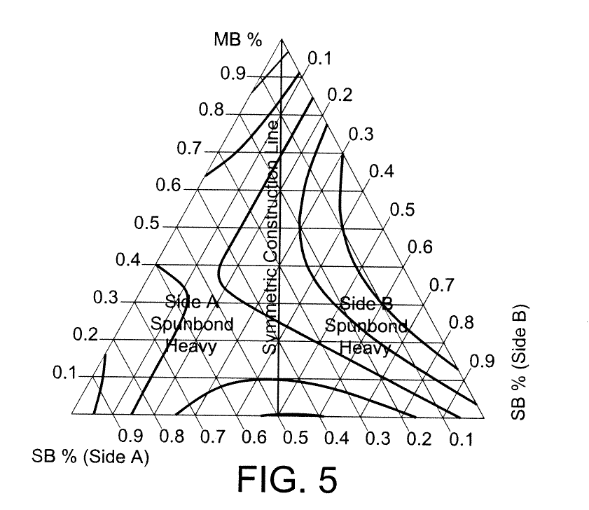

Surprisingly, durability is increased further by changing from a symmetric SMS construction to that of an asymmetric construction. More specifically, greater durability is gained when SB (cart shelf or probe side) percentage is greater than the SB percentage on the opposing side. In contrast, durability is lost when the SB (cart shelf or probe side) percentage is less than the SB percentage on the opposing side. In addition, when used as a gown or drape fabric with the heavier spunbond side outward, it is believed that the tie will be more firmly attached to the gown or the attachment mechanism will secure more durably to the drape. These generalities are illustrated by FIG. 5. Note the “symmetric construction line” representing an SMS laminate having equal weights of spunbond on either side. Also note the region to the right which represents asymmetric SMS structures, as well as a progressively more durable construction laminate.

TABLE 4
Symmetric and asymmetric SMS samples tested via Sliding Compression

| | | | | | | | SB | Sliding |
|---|---|---|---|---|---|---|---|---|
| | | | | SB | | | (probe | Com- |
| Sam- | Tar- | | | (probe | SB | MB | side) | pression |
| ple | get | SB % | MB % | side) % | (osy) | (osy) | (osy) | (g) |
| 1 | 1.70 | 69% | 19% | 12% | 1.17 | 0.32 | 0.20 | 380 |
| | | | | | | | | 580 |
| | | | | | | | | 1060 |
| 2 | 1.70 | 12% | 19% | 69% | 0.20 | 0.32 | 1.17 | 1320 |
| | | | | | | | | 1120 |
| | | | | | | | | 1060 |
| 3 | 1.70 | 65% | 23% | 12% | 1.11 | 0.39 | 0.20 | 690 |
| | | | | | | | | 620 |
| | | | | | | | | 630 |
| 4 | 1.70 | 12% | 23% | 65% | 0.20 | 0.39 | 1.11 | 820 |
| | | | | | | | | 710 |
| | | | | | | | | 790 |
| 5 | 1.85 | 68% | 21% | 11% | 1.26 | 0.39 | 0.20 | 850 |
| | | | | | | | | 650 |
| | | | | | | | | 1060 |
| 6 | 1.85 | 11% | 21% | 68% | 0.20 | 0.39 | 1.26 | 930 |
| | | | | | | | | 990 |
| | | | | | | | | 1370 |
| 7 | 1.70 | 38% | 25% | 38% | 0.64 | 0.42 | 0.64 | 1050 |
| | | | | | | | | 520 |
| | | | | | | | | 890 |
| | | | | | | | | 810 |
| | | | | | | | | 650 |
| | | | | | | | | 720 |
| 8 | 1.85 | 37% | 25% | 37% | 0.70 | 0.47 | 0.70 | 1090 |
| | | | | | | | | 1090 |
| | | | | | | | | 890 |
| | | | | | | | | 680 |
| | | | | | | | | 980 |
| | | | | | | | | 1000 |
| 9 | 1.85 | 64% | 26% | 11% | 1.17 | 0.47 | 0.20 | 1160 |
| | | | | | | | | 780 |
| | | | | | | | | 700 |
| 10 | 1.85 | 11% | 26% | 64% | 0.20 | 0.47 | 1.17 | 690 |
| | | | | | | | | 1030 |
| | | | | | | | | 1190 |

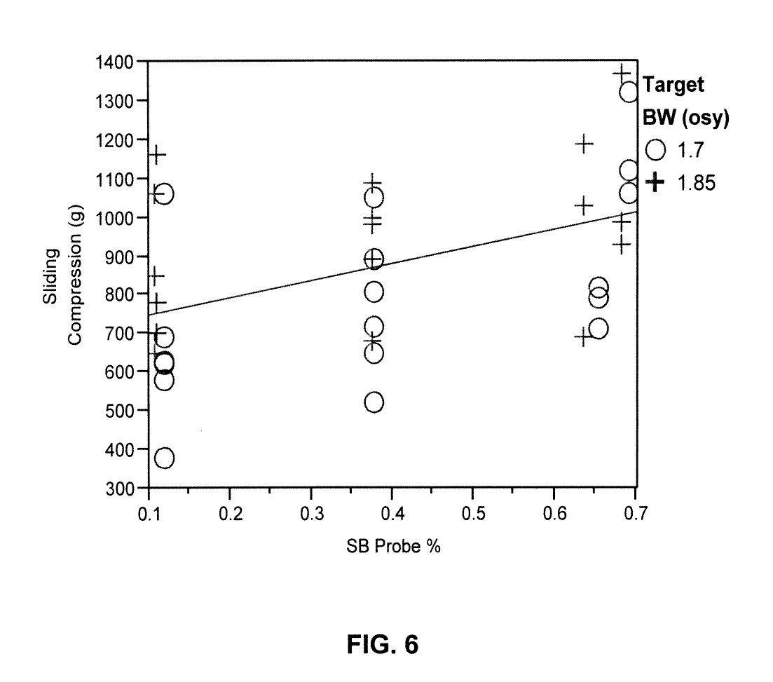

Sliding compression results, presented in Table 4, plotted as a function of SB percentage oriented towards the probe (i.e., SB Probe percentage) are shown in FIG. 6. A linear fit of the data is also plotted in FIG. 6, illustrating the general increase in durability as a function of SB Probe percentage.

Table 5 contains parameter estimates of equation of the linear fit presented in FIG. 6. In FIG. 6, the Sliding Compression results are plotted on Y axis as a function of overall weight percentage of the SMS laminate that is comprised of spunbond oriented toward the probe (X axis). Note that the overall weight percentage of the SMS laminate that is comprised of spunbond oriented towards the probe (i.e., SB Probe percentage) is found to be a statistically significant effect.

TABLE 5

| Term | Estimate | Std Error | t Ratio | Prob > \|t\| |
|---|---|---|---|---|
| Intercept | 706.04988 | 69.41786 | 10.17 | <.0001* |
| SB Probe % | 442.76503 | 155.5839 | 2.85 | 0.0075* |

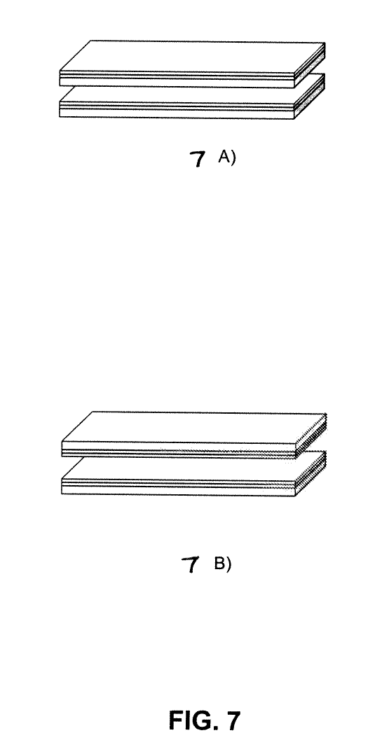

Asymmetric SMS laminate can also be used in multiple SMS sheet form. In multiple sheet form the asymmetric SMS sheets can be co-oriented with one another or counter-oriented with one another. This is illustrated in FIGS. 7A and 7B, which shows a cross-sectional view of a co-oriented (FIG. 7A) and counter oriented (FIG. 7B) SMS laminate. By “co-oriented” it is meant that the heavier basis weight side of the layers is facing the same direction.

In the disclosed wrap each SMS laminate desirably has a basis weight of between about 17 and 119 gsm. Other embodiments may have a basis weight between about 34 and 87 gsm and still others may have a basis weight between 17 and 30 gsm.

The SMS laminate wrap may have its construction skewed so that the first spunbond layer contains between 40 and 80 percent of the basis weight of the laminate. Alternatively, the SMS laminate may have a first spunbond layer that has between 50 and 70 percent of the basis weight of the laminate. The SMS wrap may have a meltblown layer having between 10 and 40 percent of the basis weight of the laminate.

In addition to durability, a medical fabric must have sufficient permeability to allow the sterilization gas to pass freely through it. The permeability of the wrap material may range from 10 to about 500 cubic feet per minute (CFM) as characterized in terms of Frazier permeability. For example, the permeability of the wrap material may range from 10 to about 400 cubic feet per minute. The Frazier permeability, which expresses the permeability of a material in terms of cubic feet per minute of air through a square foot of area of a surface of the material at a pressure drop of 0.5 inch of water (or 125 Pa), was determined utilizing a Frazier Air Permeability Tester available from the Frazier Precision Instrument Company and measured in accordance with Federal Test Method 5450, Standard No. 191 A. When the wrap material may be determined generally in accordance with ISO 9237:1995 (measured with an automated air permeability machine using a 38 cm² head at a test pressure of 125 Pa, an exemplary air permeability machine is TEXTEST FX 3300 available from TEXTEST AG, Switzerland). If multiple plies or layers of SMS material are used to provide basis weights ranging from about 2 osy (67 gsm) to about 5 osy (167 gsm), the permeability of the wrap material may range from about 10 cubic feet per minute to about 30 cubic feet per minute when determined generally in accordance with ISO 9237:1995.

As used herein and in the claims, the term “comprising” is inclusive or open-ended and does not exclude additional unrecited elements, compositional components, or method steps.

While the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

We claim:

1. A medical fabric that comprises a laminate having a first spunbond layer, a meltblown layer and a second spunbond layer, wherein said first spunbond layer has a greater basis weight than said second spunbond layer and wherein said fabric is used as a sterilization wrap, gown or drape.

2. The wrap of claim 1 wherein said laminate has a basis weight of between about 17 and 119 gsm.

3. The wrap of claim 1 wherein said laminate has a basis weight of between about 34 and 87 gsm.

4. The wrap of claim 1 wherein said first spunbond layer has between 40 and 85 percent of the basis weight of the laminate.

5. The wrap of claim 1 wherein said first spunbond layer has between 50 and 70 percent of the basis weight of the laminate.

6. The wrap of claim 1 wherein said meltblown layer has between 10 and 30 percent of the basis weight of the laminate.

7. The wrap of claim 1 having a Frazier permeability of between 10 and 30 cubic feet per minute (CFM).

8. A medical fabric that comprises a laminate having a first spunbond layer, a meltblown layer and a second spunbond layer, wherein said first spunbond layer has a greater basis weight than said second spunbond layer, the first spunbond layer being oriented away from an item to be sterilized, a patient or medical professional.

9. The wrap of claim 8 wherein said laminate has a basis weight of between about 17 and 119 gsm.

10. The wrap of claim 8 wherein said laminate has a basis weight of between about 34 and 87 gsm.

11. The wrap of claim 8 wherein said first spunbond layer has between 40 and 85 percent of the basis weight of the laminate.

12. The wrap of claim 8 wherein said first spunbond layer has between 50 and 70 percent of the basis weight of the laminate.

13. The wrap of claim 8 wherein said meltblown layer has between 10 and 30 percent of the basis weight of the laminate.

14. A multilayer sterilization wrap having at least two SMS layers, the first SMS layer on a side away from an item to be sterilized having a basis weight between 17 and 87 gsm and having an asymmetrical construction with an outer spunbond layer having a greater basis weight than an inner spunbond layer.

15. The multilayer sterilization wrap of claim 15 wherein a second SMS layer has a symmetrical construction wherein both spunbond layers have the same basis weight.

16. The multilayer sterilization wrap of claim 15 wherein a second SMS layer has an asymmetrical construction wherein the spunbond layers have different basis weights.

17. The multilayer sterilization wrap of claim 15 wherein the spunbond layer of the second SMS layer having the greater basis weight is on a side away from an item to be sterilized.

18. The multilayer sterilization wrap of claim 15 wherein the spunbond layer of the second SMS layer having the lower basis weight is on a side away from an item to be sterilized.

19. The multilayer sterilization wrap of claim 14 wherein the laminate is made in the sequential manner wherein individual layers are deposited onto a moving forming belt; first a spunbond fabric layer, then a meltblown fabric layer and last another spunbond layer, and bonded to form said laminate.
